(19) United States
(12) Patent Application Publication
(10) Pub. No.: US 2009/0203709 A1
(43) Pub. Date: Aug. 13, 2009

(54) Pharmaceutical Dosage Form For Oral Administration Of Tyrosine Kinase Inhibitor

(75) Inventors: Joyce L. Steinberg, Northbrook, IL (US); Neeraj Gupta, Waukegan, IL (US); Rajendra S. Pradhan, Buffalo Grove, IL (US); Sari H. Enschede, River Forest, IL (US); Rod A. Humerickhouse, Highland Park, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/365,966

(22) Filed: Feb. 5, 2009

Related U.S. Application Data
(60) Provisional application No. 61/026,975, filed on Feb. 7, 2008.

(51) Int. Cl. A61K 31/416; A61K 31/506; A61P 35/00; A61K 31/517; A61K 31/44; A61K 47/08; A61K 47/14

(52) U.S. Cl. 514/252.18; 514/252.19; 514/266.22; 514/266.24; 514/350; 514/406; 514/772; 514/785

(57) ABSTRACT

A pharmaceutical dosage form comprises a solid dispersion product of at least one tyrosine kinase inhibitor, at least one pharmaceutically acceptable polymer, and at least one pharmaceutically acceptable solubilizer.

### CROSS REFERENCE TO RELATED APPLICATION

The present application claims priority to U.S. Provisional Application Ser. No. 61/026,975, filed Feb. 7, 2008; hereby incorporated in entirety by reference.

### FIELD OF THE INVENTION

The present invention relates to a pharmaceutical dosage form for oral administration of tyrosine kinase inhibitors, a method of preparing the dosage form and a method of treating proliferative disorders.

### BACKGROUND OF THE INVENTION

Tyrosine kinase inhibiting compounds are useful for treating diseases caused or exacerbated by upregulation or overexpression of protein tyrosine kinases. Unfortunately, the crystalline forms of many known tyrosine kinase inhibitors are characterized by a more or less pronounced poor solubility in aqueous liquids which affects their dissolution rate and bioavailability.

A measure of the potential usefulness of an oral dosage form of a pharmaceutical agent is the bioavailability observed after oral administration of the dosage form. Various factors can affect the bioavailability of a drug when administered orally. These factors include aqueous solubility, drug absorption throughout the gastrointestinal tract, dosage strength and first-pass effect. Aqueous solubility is one of the most important of these factors.

For a variety of reasons, such as patient compliance and taste masking, a solid dosage form is usually preferred over a liquid dosage form. In most instances, however, oral solid dosage forms of a drug provide a lower bioavailability than oral solutions of the drug.

There have been attempts to improve the bioavailability provided by solid dosage forms by forming solid solutions of the drug. Solid solutions are preferred physical systems because the components therein readily form liquid solutions when contacted with a liquid medium such as gastric juice. The ease of dissolution may be attributed at least in part to the fact that the energy required for dissolution of the components from a solid solution is less than that required for the dissolution of the components from a crystalline or microcrystalline solid phase. It is, however, important that the drug released from the solid solution remains water-solubilized in the aqueous fluids of the gastrointestinal tract; otherwise, the drug may precipitate in the gastrointestinal tract, resulting in low bioavailability.

WO 01/00175 discloses mechanically stable pharmaceutical dosage forms which are solid solutions of active ingredients in an auxiliary agent matrix. The matrix contains a homopolymer or a copolymer of N-vinyl pyrrolidone and a liquid or semi-solid surfactant.

WO 00/57854 discloses mechanically stable pharmaceutical dosage forms for peroral administration which contain at least one active compound, at least one thermoplastically mouldable, matrix-forming auxiliary and more than 10 and up to 40% by weight of a surface-active substance that has an HLB of between 2 and 18, is liquid at 20° C., or has a drop point at between 20 and 50° C.

US 2005/0208082 discloses a solubilizing composition comprising a mixture of vitamin E TPGS and linoleic acid. The solubilizing composition is used to disperse a lipophile in an aqueous phase. The lipophile may be a therapeutically effective lipophile such as lipophilic vitamins, coenzyme Q10, carotenoids, alpha-lipoic acid, essential fatty acids.

US 2005/0236236 discloses pharmaceutical compositions for administration of hydrophobic drugs, particularly steroids. The pharmaceutical compositions include a hydrophobic drug, a vitamin E substance and a surfactant. The reference claims a synergistic effect between the hydrophobic drug and the vitamin E substance.

### BRIEF DESCRIPTION OF THE DRAWINGS

FIG. 1 shows the ABT-869 mean dose-normalized concentration-time profiles for all doses from study M04-710.

FIG. 2 shows preliminary efficacy of ABT-869 of various doses and tumor types in study M04-710.

FIG. 3 shows radiographs of hepatocellular carcinoma, renal cell carcinoma and non-small cell lung cancer responses to treatment with ABT-869.

FIG. 4 are graphs showing the effect of ABT-869 on systolic and diastolic blood pressure.

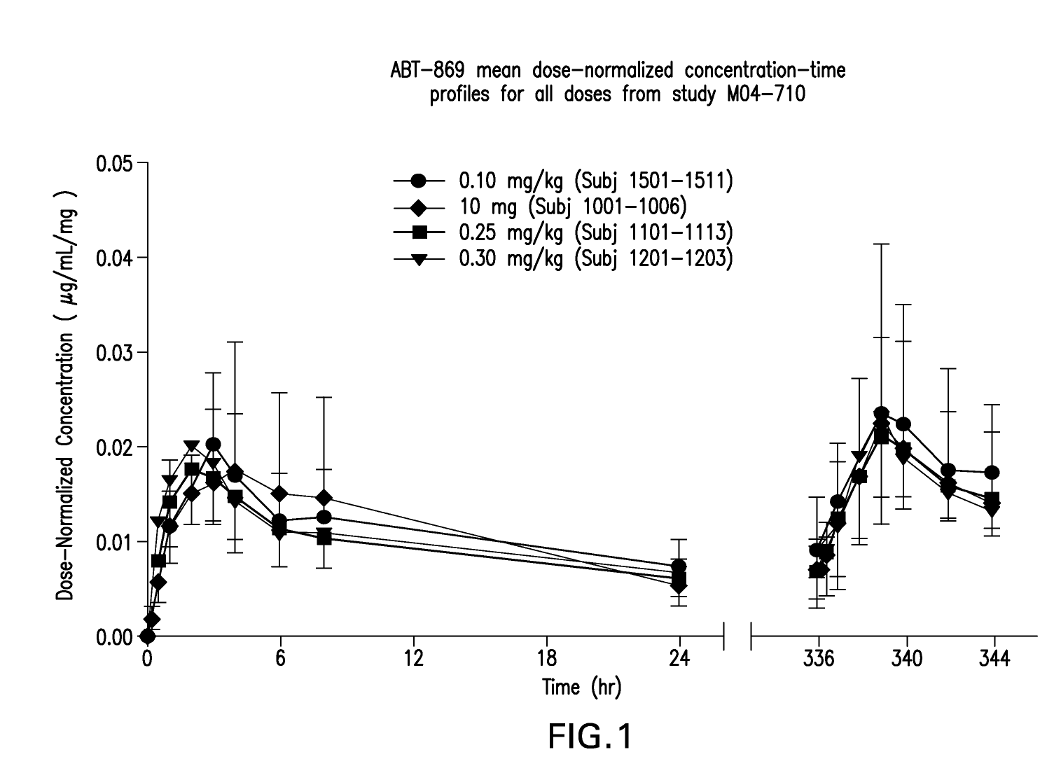

### SUMMARY OF THE INVENTION

One embodiment of this invention relates to a pharmaceutical dosage form comprising a solid dispersion product of at least one tyrosine kinase inhibitor, at least one pharmaceutically acceptable polymer, and at least one pharmaceutically acceptable solubilizer. In some embodiments of the present invention, a tyrosine kinase inhibitor is N-[4-(3-amino-1H-indazol-4-yl)phenyl]-N′-(2-fluoro-5-methylphenyl)urea (ABT 869).

Other embodiments of this invention relate to a method of treating proliferative disorders, comprising administering the dosage form containing a solid dispersion product of at least one tyrosine kinase inhibitor, at least one pharmaceutically acceptable polymer and at least one pharmaceutically acceptable solubilizer to a subject in need thereof. According to some embodiments of the invention, when the dosage form is administered to a human, a plasma profile is achieved which is characterized by a Cmax for ABT 869 from about 0.015 μg/mL/mg to about 0.027 μg/mL/mg after a single dose. According to some embodiments of the invention, when the dosage form is administered to a human patient, a plasma profile is achieved which is characterized by a Tmax for ABT 869 from 1 to about 3 hours after a single dose.

Other embodiments of this invention relate to methods for preparing a solid dosage form comprising a solid dispersion product of at least one tyrosine kinase inhibitor, at least one pharmaceutically acceptable polymer, and at least one pharmaceutically acceptable solubilizer, comprising the steps of a) preparing a homogeneous melt of said at least one tyrosine kinase inhibitor, said at least one pharmaceutically acceptable polymer and said at least one solubilizer, and allowing the melt to solidify to obtain a solid dispersion product.

### DETAILED DESCRIPTION OF THE INVENTION

There is a continuing need for the development of improved oral solid dosage forms of tyrosine kinase inhibitors.

The invention relates to a pharmaceutical dosage form which comprises a solid dispersion product of at least one tyrosine kinase inhibitor, at least one pharmaceutically acceptable polymer, and at least one pharmaceutically acceptable solubilizer.

In the dosage forms of the invention, the active ingredient is present as a solid dispersion or, preferably, as a solid solution. The term “solid dispersion” defines a system in a solid state (as opposed to a liquid or gaseous state) comprising at least two components, wherein one component is dispersed evenly throughout the other component or components. For example, the active ingredient or combination of active ingredients is dispersed in a matrix comprised of the pharmaceutically acceptable polymer(s) and pharmaceutically acceptable solubilizers. The term “solid dispersion” encompasses systems having small particles, typically of less than 1 μm in diameter, of one phase dispersed in another phase. When said dispersion of the components is such that the system is chemically and physically uniform or homogeneous throughout or consists of one phase (as defined in thermodynamics), such a solid dispersion will be called a “solid solution” or a “glassy solution”. A glassy solution is a homogeneous, glassy system in which a solute is dissolved in a glassy solvent. Glassy solutions and solid solutions are preferred physical systems. These systems do not contain any significant amounts of active ingredients in their crystalline or microcrystalline state, as evidenced by thermal analysis (DSC) or X-ray diffraction analysis (WAXS).

The dosage forms according to the invention are characterized by an excellent stability and, in particular, exhibit high resistance against recrystallization or decomposition of the active ingredient(s).

The dosage forms of the present invention exhibit a release and absorption behaviour that is characterized by high attainable AUC (area under the plasma concentration-time curve from 0 to 48 hours), high attainable Cₘₐₓ (maximum plasma concentration), and low Tₘₐₓ (time to reach maximum plasma concentration).

The term “AUC” means “Area Under the Curve” and is used in its normal meaning, i.e. as the area under the plasma concentration-time curve. “AUC₀₋₄₈” and “AUC_{0-∞}” refer to the area under the plasma concentration-time curve from 0 to 48 hours or from 0 hours to infinity, respectively.

In a preferred embodiment the invention provides a dosage form wherein said tyrosine kinase inhibitor is N-[4-(3-amino-1H-indazol-4-yl)phenyl]-N′-(2-fluoro-5-methylphenyl)-urea (ABT 869) (or a hydrate, solvate, N-oxide, or a pharmaceutically acceptable acid or base addition salt thereof). When administered to a human patient, in certain embodiments, the dosage form produces a plasma profile characterized by a Cₘₐₓ for ABT 869 from about 0.015 μg/mL/mg to about 0.027 μg/mL/mg, in particular about 0.023±0.004 μg/mL/mg (mean±SD), after a single dose.

When administered to the human patient, in certain embodiments, the dosage form produces a plasma profile characterized by a Tₘₐₓ for ABT 869 of about 1 to about 3 hours, in particular about 2.8±0.6 hours, after a single dose.

In particular embodiments, when administered to the human patient, the dosage form produces an AUC₀₋₄₈ per mg of ABT 869 from about 0.23 μg*hr/mL/mg to about 0.56 μg*hr/mL/mg, in particular about 0.40±0.10 μg*h/mL/mg, or an AUC_{0-∞} per mg of ABT 869 from about 0.27 μg*hr/mL/mg to about 0.81 μg*hr/mL/mg, in particular about 0.55±0.17 μg*h/mL/mg, per mg of dose after a single dose.

The plasma concentration profile may suitably be established in a group of at least ten healthy humans under fasting conditions, based on blood sampling at 0, 1, 3, 4, 6, 8, 24 and 48 hours. “Fasting conditions” means that the patients abstain from food or drink consumption except water and concomitant medications for 2 hours prior to and after dosing. Once the concentration-time points have been determined, the plasma concentration profile may be calculated, e.g. by a computer program or by the trapezoidal method. Administration of single dose of 10 mg ABT 869 to a human is considered suitable for determining the AUC values as used herein.

A preferred feature of the dosage form is their ability to release fine particles having, e.g., an average particle size of less than about 1000 nm, preferably less than about 800 nm, in particular less than about 500 nm and especially preferred less than about 200 nm, when the dosage form is brought into contact with an aqueous liquid. The fine particles contain solubilised tyrosine kinase inhibitor, preferably in an essentially non-crystalline state. When the dosage form is administered orally, the aqueous liquid will be gastric juices. For in vitro testing purposes, the aqueous liquid may suitably be a volume of 900 ml of 1 N hydrochloric acid (USP apparatus II).

The dispersion formed upon contact with an aqueous liquid may also be useful as such, for example as oral liquid dosage form or parenteral injections.

Generally, the solid dispersion product comprises
from about 0.5 to 40% by weight, preferably from about 1 to 25% by weight, of said at least one tyrosine kinase inhibitor, from about 40 to 97.5% by weight, preferably from about 50 to 94% by weight, of said at least one pharmaceutically acceptable polymer, from about 2 to 20% by weight, preferably from about 5 to 20% by weight, of said at least one solubilizer, and from about 0 to 15% by weight, preferably from about 0 to 10% by weight, of additives.

Whereas the dosage form of the invention may consist entirely of solid dispersion product, additives and adjuvants are usually used in formulating the solid dispersion product into the dosage forms. Generally, the dosage form comprises at least 10% by weight, preferably at least 40% by weight, and most preferred at least 45% by weight, of solid dispersion product, based on the total weight of the solid dosage form.

Typically, a single dosage form of the invention contains the equivalent of about 0.1 mg to about 100 mg, preferably about 1.0 mg to about 50 mg, in particular 2.5 mg to 25 mg, of said at least one tyrosine kinase inhibitor.

The inventive dosage form comprises a tyrosine kinase inhibitor or a combination of two or more tyrosine kinase inhibitors. The dosage form may comprise a combination of one or more tyrosine kinase inhibitors and at least one further active ingredient. Various kinds of tyrosine kinase inhibitors can be effectively utilized.

A preferred tyrosine kinase inhibitor is ABT 869 [N-[4-(3-1H-indazol-4-yl)phenyl]-N′-(2-fluoro-5-methylphenyl)urea] the preparation of which is described in WO 04/113304. The molecular structure of ABT 869 is depicted below:

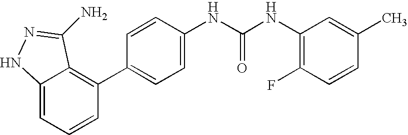

A further preferred tyrosine kinase inhibitor is N-(4-(4-aminothieno[2,3-d]pyrimidin-5-yl)phenyl)-N′-(2-fluoro-5-(trifluoromethyl)phenyl)urea the preparation of which is described in US 2007/0155758.

Further tyrosine kinase inhibitors which may be used include sorafenib (trade name Nexavar), dasatinib, lapatinib (trade name Tykerb), imatinib (trade name Gleevec), motesanib, vandetanib (Zactima), MP-412, lestaurtinib, XL647, XL999, tandutinib, PKC412, nilotinib, AEE788, OSI-930, OSI-817, sunitinib maleate (trade name Sutent) and axitinib.

The term “tyrosine kinase inhibitors” is intended to encompass the hydrates, solvates (such as alcoholates), N-oxides, pharmaceutically acceptable acid or base addition salts of tyrosine kinase inhibiting compounds.

Pharmaceutically acceptable acid addition salts comprises the acid addition salt forms which can be obtained conveniently by treating the base form of the active ingredient with appropriate organic and inorganic acids.

Active ingredients containing an acidic proton may be converted into their non-toxic metal or amine addition salt forms by treatment with appropriate organic and inorganic bases.

The invention is particularly useful for water-insoluble or poorly water soluble (or “hydrophobic” or “lipophilic” compounds. Compounds are considered water-insoluble or poorly water-soluble when their solubility in water 25° C. is less than 1 g/100 ml, especially less than 0.1 g/100 ml.

The term “pharmaceutically acceptable solubilizer” as used herein refers to a pharmaceutically acceptable non-ionic surfactant. The solubilizer may effectuate an instantaneous emulsification of the active ingredient released from the dosage form and/or prevent precipitation of the active ingredient in the aqueous fluids of the gastrointestinal tract. A single solubilizer as well as combinations of solubilizers may be used. According to an embodiment of the invention, the solid dispersion product comprises a combination of two or more pharmaceutically acceptable solubilizers.

Preferred solubilizers are selected from sorbitan fatty acid esters, polyalkoxylated fatty acid esters such as, for example, polyalkoxylated glycerides, polyalkoxylated sorbitan fatty acid esters or fatty acid esters of polyalkylene glycols, polyalkoxylated ethers of fatty alcohols, tocopheryl compounds or mixtures of two or more thereof. A fatty acid chain in these compounds ordinarily comprises from 8 to 22 carbon atoms. The polyalkylene oxide blocks comprise on average from 4 to 50 alkylene oxide units, preferably ethylene oxide units, per molecule.

Suitable sorbitan fatty acid esters are sorbitan monolaurate, sorbitan monopalmitate, sorbitan monostearate (Span® 60), sorbitan monooleate (Span® 80), sorbitan tristearate, sorbitan trioleate, sorbitan monostearate, sorbitan monolaurate or sorbitan monooleate.

Examples of suitable polyalkoxylated sorbitan fatty acid esters are polyoxyethylene (20) sorbitan monolaurate, polyoxyethylene (20) sorbitan monopalmitate, polyoxyethylene (20) sorbitan monostearate, polyoxyethylene (20) sorbitan monooleate (Tween® 80), polyoxyethylene (20) sorbitan tristearate (Tween® 65), polyoxyethylene (20) sorbitan trioleate (Tween® 85), polyoxyethylene (4) sorbitan monostearate, polyoxyethylene (4) sorbitan monolaurate or polyoxyethylene (4) sorbitan monooleate.

Suitable polyalkoxylated glycerides are obtained for example by alkoxylation of natural or hydrogenated glycerides or by transesterification of natural or hydrogenated glycerides with polyalkylene glycols. Commercially available examples are polyoxyethylene glycerol ricinoleate 35, polyoxyethylene glycerol trihydroxystearate 40 (Cremophor® RH40, BASF AG) and polyalkoxylated glycerides like those obtainable under the proprietary names Gelucire® and Labrafil® from Gattefosse, e.g. Gelucire® 44/14 (lauroyl macrogol 32 glycerides prepared by transesterification of hydrogenated palm kernel oil with PEG 1500), Gelucire® 50/13 (stearoyl macrogol 32 glycerides, prepared by transesterification of hydrogenated palm oil with PEG 1500) or Labrafil M1944 CS (oleoyl macrogol 6 glycerides prepared by transesterification of apricot kernel oil with PEG 300).

A suitable fatty acid ester of polyalkylene glycols is, for example, PEG 660 hydroxy-stearic acid (polyglycol ester of 12-hydroxystearic acid (70 mol %) with 30 mol % ethylene glycol).

Suitable polyalkoxylated ethers of fatty alcohols are, for example, PEG (2) stearyl ether (Brij® 72), macrogol 6 cetylstearyl ether or macrogol 25 cetylstearyl ether.

In general, the tocopheryl compound corresponds to the formula below

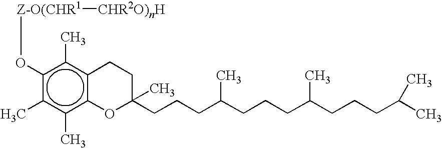

wherein Z is a linking group, R¹ and R² are, independently of one another, hydrogen or C1-C4 alkyl and n is an integer from 5 to 100, preferably 10 to 50. Typically, Z is the residue of an aliphatic dibasic acid such as glutaric, succinic, or adipic acid. Preferably, both R¹ and R² are hydrogen.

It was found that solubilizers or combination of solubilizers having a defined HLB (hydrophilic lipophilic balance) value are preferred over other solubilizers.

The HLB system (Fiedler, H. B., Encyclopedia of Excipients, 5^{th} ed., Aulendorf: ECV-Editio-Cantor-Verlag (2002)) attributes numeric values to surfactants, with lipophilic substances receiving lower HLB values and hydrophilic substances receiving higher HLB values.

Where a single solubilizer is employed it suitably has an HLB value of from 3.5 to 13, preferably from 4 to 11.

Where a combination of two or more pharmaceutically acceptable solubilizers is used the combination of pharmaceutically acceptable solubilizers suitably has an averaged HLB value in the range of from 4.5 to 12, preferably 5 to 11. The averaged HLB value may be computed by multiplying the HLB value of each individual solubilizer by the proportion of the individual solubilizer with regard to the total amount of solubilizers present and adding together the contributions of the individual solubilizers.

Quite unexpectedly, a combination of at least one solubilizer having a relatively high HLB value and at least one solubilizer having a relatively low HLB value proved particularly useful. The high HLB solubilizer suitably has an HLB value in the range of from 8 to 15, preferably 10 to 14. The low HLB solubilizer suitably has an HLB value in the range of from 3 to 6, preferably 3.5 to 5. The weight ratio of high HLB solubilizer and low HLB solubilizer may be in the range of from 9:1 to 1:9, preferably 5:1 to 1:5.

Solubilizers having an HLB value in the range of from 8 to 15 may be selected from Cremophor® RH40 (HLB 13), Tween® 65 (HLB 10.5), Tween® 85 (HLB 11) Preferred high HLB solubilizers are tocopheryl compounds having a polyalkylene glycol moiety.

The preferred tocopheryl compound is alpha tocopheryl polyethylene glycol succinate, which is commonly abbreviated as vitamin E TPGS. Vitamin E TPGS is a water-soluble form of natural-source vitamin E prepared by esterifying d-alpha-tocopheryl acid succinate with polyethylene glycol 1000. Vitamin E TPGS is available from Eastman Chemical Company, Kingsport, Tenn., USA and is listed in the US pharmacopoeia (NF).

Solubilizers having an HLB value in the range of from 3 to 6 may be selected from Span® (HLB 4.7), Span® 80 (HLB 4.3), Labrafil M1944 CS (HLB 4.0) and Brij® 72 (HLB 4.9).

A preferred low HLB solubilizer is an alkylene glycol fatty acid monoester or a mixture of alkylene glycol fatty acid mono- and diester.

The preferred alkylene glycol fatty acid mono ester is a propylene glycol fatty acid mono ester, such as propylene glycol monolaurate (available under the trade name LAUROGLYCOL® from Gattefosse, France). Commercially available propylene glycol lauric acid mono ester products consist of a mixture of mono- and dilaurate. Two propylene glycol monolaurate products are specified in the European Pharmacopoea (referenced “type I” and “type II” respectively). Both types are suitable for carrying out the present invention, with propylene glycol monolaurate “type I” being the most preferred. This “type I” product having a HLB value of about 4 consists of a mixture having between 45 and up to 70% mono-laurate and between 30 and up to 55% of di-laurate. The “type II” product is specified according to Pharm. Eur. as having a minimum of 90% mono-laurate and a maximum of 10% of di-laurate.

Where a mixture of alkylene glycol fatty acid mono and diester is employed, this preferably contains at least 40% by weight of the mono ester, especially 45 to 95% by weight, relative to the weight of the ester mixture.

Thus, in a preferred embodiment, the combination of solubilizers comprises (i) at least one tocopheryl compound having a polyalkylene glycol moiety, preferably alpha to copheryl polyethylene glycol succinate, and (ii) at least one alkylene glycol fatty acid monoester or a mixture of alkylene glycol fatty acid mono- and diester.

The pharmaceutically acceptable polymer may be selected from water-soluble polymers, water-dispersible polymers or water-swellable polymers or any mixture thereof. Polymers are considered water-soluble if they form a clear homogeneous solution in water. When dissolved at 20° C. in an aqueous solution at 2% (w/v), the water-soluble polymer preferably has an apparent viscosity of 1 to 5000 mPa·s, more preferably of 1 to 700 mPa·s, and most preferably of 5 to 100 mPa·s. Water-dispersible polymers are those that, when contacted with water, form colloidal dispersions rather than a clear solution. Upon contact with water or aqueous solutions, water-swellable polymers typically form a rubbery gel.

Preferably, the pharmaceutically acceptable polymer employed in the invention has a Tg of at least 40° C., preferably at least +50° C., most preferably from 80° to 180. ° C. “Tg” means glass transition temperature. Methods for determining Tg values of the organic polymers are described in “Introduction to Physical Polymer Science”, 2nd Edition by L.H. Sperling, published by John Wiley & Sons, Inc., 1992. The Tg value can be calculated as the weighted sum of the Tg values for homopolymers derived from each of the individual monomers, i, that make up the polymer: Tg=ΣWᵢXᵢ where W is the weight percent of monomer i in the organic polymer, and X is the Tg value for the homopolymer derived from monomer i. Tg values for the homopolymers may be taken from “Polymer Handbook”, 2nd Edition by J. Brandrup and E. H. Immergut, Editors, published by John Wiley & Sons, Inc., 1975.

Various additives contained in the solid dispersion product or even the active ingredient(s) itself may exert a plasticizing effect on the polymer and thus depress the Tg of the polymer such that the final solid dispersion product has a somewhat lower Tg than the starting polymer used for its preparation. In general, the final solid dispersion product has a Tg of 10° C. or higher, preferably 15° C. or higher, more preferably 20° C. or higher and most preferred 30° C. or higher.

For example, preferred pharmaceutically acceptable polymers can be selected from the group comprising homopolymers and copolymers of N-vinyl lactams, especially homopolymers and copolymers of N-vinyl pyrrolidone, e.g. polyvinylpyrrolidone (PVP), copolymers of N-vinyl pyrrolidone and vinyl acetate or vinyl propionate, cellulose esters and cellulose ethers, in particular methylcellulose and ethylcellulose, hydroxyalkylcelluloses, in particular hydroxypropylcellulose, hydroxyalkylalkylcelluloses, in particular hydroxypropylmethylcellulose, cellulose phthalates or succinates, in particular cellulose acetate phthalate and hydroxypropylmethylcellulose phthalate, hydroxypropylmethylcellulose succinate or hydroxypropylmethylcellulose acetate succinate; high molecular polyalkylene oxides such as polyethylene oxide and polypropylene oxide and copolymers of ethylene oxide and propylene oxide, polyvinyl alcohol-polyethylene glycol-graft copolymers (available as Kollicoat® IR from BASF AG, Ludwigshafen, Germany); polyacrylates and polymethacrylates such as methacrylic acid/ethyl acrylate copolymers, methacrylic acid/methyl methacrylate copolymers, butyl methacrylate/2-dimethylaminoethyl methacrylate copolymers, poly(hydroxyalkyl acrylates), poly(hydroxyalkyl methacrylates), polyacrylamides, vinyl acetate polymers such as copolymers of vinyl acetate and crotonic acid, partially hydrolyzed polyvinyl acetate (also referred to as partially saponified “polyvinyl alcohol”), polyvinyl alcohol, oligo- and polysaccharides such as carrageenans, galactomannans and xanthan gum, or mixtures of one or more thereof.

Among these, homopolymers or copolymers of N-vinyl pyrrolidone, in particular a co-polymer of N-vinyl pyrrolidone and vinyl acetate, are preferred. A particularly preferred polymer is a copolymer of 60% by weight of the copolymer, N-vinyl pyrrolidone and 40% by weight of the copolymer, vinyl acetate.

A further polymer which can be suitably used is Kollidon® SR (available from BASF AG, Ludwigshafen, Germany) which comprises a mixture of PVP and polyvinylacetate.

The solid dispersion product may be prepared by a variety of methods. The solid dispersion product may be prepared by a solvent evaporation method. In a solvent evaporation method, the at least one tyrosine kinase inhibitor, the at least one pharmaceutically acceptable polymer and the at least one pharmaceutically acceptable solubilizer are dissolved in a common solvent or combination of solvents and the solvents are removed from the solution obtained by evaporation.

Preferably, the solid dispersion product is prepared by melt-extrusion. The melt-extrusion process comprises the steps of preparing a homogeneous melt of the active ingredient or the combination of active ingredients, the pharmaceutically acceptable polymer and the solubilizers, and cooling the melt until it solidifies. “Melting” means a transition into a liquid or rubbery state in which it is possible for one component to become homogeneously embedded in the other. Typically, one component will melt and the other components will dissolve in the melt, thus forming a solution. Melting usually involves heating above the softening point of the pharmaceutically acceptable polymer. The preparation of the melt can take place in a variety of ways. The mixing of the components can take place before, during or after the formation of the melt. For example, the components can be mixed first and then melted or simultaneously mixed and melted. Usually, the melt is homogenized in order to disperse the active ingredients efficiently. Also, it may be convenient first to melt the pharmaceutically acceptable polymer and then to admix and homogenize the active ingredients.

Usually, the melt temperature is in the range of 70 to 250° C., preferably 80 to 180° C., most preferably 100 to 140° C.

The active ingredients can be employed as such or as a solution or dispersion in a suitable solvent such as alcohols, aliphatic hydrocarbons or esters. Another solvent which can be used is liquid carbon dioxide. The solvent is removed, e.g. evaporated, upon preparation of the melt.

Various additives may be included in the melt, for example flow regulators such as colloidal silica; lubricants, bulking agents (fillers), disintegrants, plasticizers, stabilizers such as antioxidants, light stabilizers, radical scavengers, or stabilizers against microbial attack.

The melting and/or mixing takes place in an apparatus customary for this purpose. Particularly suitable are extruders or kneaders. Suitable extruders include single screw extruders, intermeshing screw extruders or else multiscrew extruders, preferably twin screw extruders, which can be corotating or counterrotating and, optionally, equipped with kneading disks or other screw elements for mixing or dispersing the melt. It will be appreciated that the working temperatures will also be determined by the kind of extruder or the kind of configuration within the extruder used. Part of the energy needed to melt, mix and dissolve the components in the extruder can be provided by heating elements. However, the friction and shearing of the material in the extruder may also provide a substantial amount of energy to the mixture and aid in the formation of a homogeneous melt of the components.

The extrudate exiting from the extruder ranges from pasty to viscous. Before allowing the extrudate to solidify, the extrudate may be directly shaped into virtually any desired shape. Shaping of the extrudate may be conveniently carried out by a calendar with two counter-rotating rollers with mutually matching depressions on their surface. A broad range of tablet forms can be attained by using rollers with different forms of depressions. If the rollers do not have depressions on their surface, films can be obtained. Alternatively, the extrudate is moulded into the desired shape by injection-moulding. Alternatively, the extrudate is subjected to profile extrusion and cut into pieces, either before (hot-cut) or after solidification (cold-cut).

Additionally, foams can be formed if the extrudate contains a propellant such as a gas, e.g. carbon dioxide, or a volatile compound, e.g. a low molecular-weight hydrocarbon, or a compound that is thermally decomposable to a gas. The propellant is dissolved in the extrudate under the relatively high pressure conditions within the extruder and, when the extrudate emerges from the extruder die, the pressure is suddenly released.

Thus the solvability of the propellant is decreased and/or the propellant vaporises so that a foam is formed.

Optionally, the resulting solid solution product is milled or ground to granules. The granules may then be filled into capsules or may be compacted. Compacting means a process whereby a powder mass comprising the granules is densified under high pressure in order to obtain a compact with low porosity, e.g. a tablet. Compression of the powder mass is usually done in a tablet press, more specifically in a steel die between two moving punches.

At least one additive selected from flow regulators, disintegrants, bulking agents (fillers) and lubricants is preferably used in compacting the granules. Disintegrants promote a rapid disintegration of the compact in the stomach and keep the liberated granules separate from one another. Suitable disintegrants are crosslinked polymers such as crosslinked polyvinyl pyrrolidone and crosslinked sodium carboxymethyl cellulose. Suitable bulking agents (also referred to as “fillers”) are selected from lactose, calcium hydrogenphosphate, microcrystalline cellulose (Avicel®), magnesium oxide, potato or corn starch, isomalt, polyvinyl alcohol.

Suitable flow regulators are selected from highly dispersed silica (Aerosil®), and animal or vegetable fats or waxes.

A lubricant is preferably used in compacting the granules. Suitable lubricants are selected from polyethylene glycol (e.g., having a Mw of from 1000 to 6000), magnesium and calcium stearates, sodium stearyl fumarate, talc, and the like.

Various other additives may be used, for example dyes such as azo dyes, organic or inorganic pigments such as aluminium oxide or titanium dioxide, or dyes of natural origin; stabilizers such as antioxidants, light stabilizers, radical scavengers, or stabilizers against microbial attack.

Dosage forms according to the invention may be provided as dosage forms consisting of several layers, for example laminated or multilayer tablets. They can be in open or closed form. “Closed dosage forms” are those in which one layer is completely surrounded by at least one other layer. Multilayer forms have the advantage that two active ingredients which are incompatible with one another can be processed, or that the release characteristics of the active ingredient(s) can be controlled. For example, it is possible to provide an initial dose by including an active ingredient in one of the outer layers, and a maintenance dose by including the active ingredient in the inner layer(s). Multilayer tablets types may be produced by compressing two or more layers of granules. Alternatively, multilayer dosage forms may be produced by a process known as “coextrusion”. In essence, the process comprises the preparation of at least two different melt compositions as explained above, and passing these molten compositions into a joint coextrusion die. The shape of the coextrusion die depends on the required drug form. For example, dies with a plain die gap, called slot dies, and dies with an annular slit are suitable.

In order to facilitate the intake of such a dosage form by a mammal, it is advantageous to give the dosage form an appropriate shape. Large tablets that can be swallowed comfortably are therefore preferably elongated rather than round in shape.

A film coat on the tablet further contributes to the ease with which it can be swallowed. A film coat also improves taste and provides an elegant appearance. If desired, the film coat may be an enteric coat. The film coat usually includes a polymeric film-forming material such as hydroxypropyl methylcellulose, hydroxypropyl cellulose, and acrylate or methacrylate copolymers. Besides a film-forming polymer, the film coat may further comprise a plasticizer, e.g. polyethylene glycol, a surfactant, e.g. a Tween® type, and optionally a pigment, e.g. titanium dioxide or iron oxides. The film-coating may also comprise talc as anti-adhesive. The film coat usually accounts for less than about 5% by weight of the dosage form.

The dosage forms of the invention are useful for treating proliferative disorders, especially tumors or cancers. The proliferative disorder may be selected from the group consisting of neurofibromatosis, tuberous sclerosis, hemangiomas and lymphangiogenesis, cervical, anal and oral cancers, eye or ocular cancer, stomach cancer, colon cancer, bladder cancer, rectal cancer, liver cancer, pancreas cancer, lung cancer, breast cancer, cervix uteri cancer, corpus uteri cancer, ovary cancer, prostate cancer, testis cancer, renal cancer, brain cancer, cancer of the central nervous system, head and neck cancer, throat cancer, skin melanoma, acute lymphocytic leukemia, acute myelogenous leukemia, Ewing's Sarcoma, Kaposi's Sarcoma, basal cell carcinoma and squamous cell carcinoma, small cell lung cancer, choriocarcinoma, rhabdomyosarcoma, angiosarcoma, hemangioendothelioma, Wilms Tumor, neuroblastoma, mouth/pharynx cancer, esophageal cancer, larynx cancer, lymphoma, multiple myeloma; cardiac hypertrophy, age-related macular degeneration and diabetic retinopathy.

The exact dose and frequency of administration depends on the particular condition being treated, the age, weight and general physical condition of the particular patient as well as other medication the individual may be taking, as is well known to those skilled in the art.

The following examples will serve to further illustrate the invention without limiting it.

### Example 1

### Preparation of Solid Dispersion Products

Formulations of various compositions were produced as shown in Table 1 below. The active ingredient (N-[4-(3-amino-1H-indazol-4-yl)phenyl]-N′-(2-fluoro-5-methylphenyl)-urea ethanolate) was mixed in a turbula blender with a pre-granulated mixture of Kollidon VA64 (copolymer of 60% by weight N-vinyl pyrrolidone and 40% by weight vinyl acetate) and the solubilizer(s). Additionally 1% of colloidal silicon dioxide was added to improve flow properties. The powdery mixture was extruded in a Leistritz micro 18 GMP-extruder at the extrusion temperature and rotational speed as shown in table 1.

TABLE 1

| | | Kollidon | | | | |
|---|---|---|---|---|---|---|
| | ABT 869 | VA64 | Solubilizer 1 | Solubilizer 2 | | |
| Example | wt % | wt. % | wt. % | wt. % | T (° C.) | U/min |
| L | 5 | 85 | Sorbitanmonolaurate | 4.81 | 140 | 150 |
| | | | 10 | | | |
| M | 5 | 90 | Propylenglycol monolaurate** | TPGS* | 140 | 150 |
| | | | 3.1 | 1.9 | | |
| N | 5 | 80 | Sorbitanmonolaurate | none | 140 | 150 |
| | | | 15 | | | |
| O | 5 | 80 | Sorbitanmonolaurate | none | 125 | 150 |
| | | | 15 | | | |
| P | 5 | 80 | Tween 80 | none | 140 | 150 |
| | | | 15 | | | |
| Q | 5 | 80 | Tween 80 | none | 125 | 150 |
| | | | 15 | | | |
| R | 5 | 80 | Propylenglycol monolaurate** | TPGS* | 140 | 150 |
| | | | 10 | 5 | | |
| S | 5 | 80 | Propylenglycol monolaurate** | TPGS* | 125 | 150 |
| | | | 10 | 5 | | |
| A | 7.5 | 87.5 | Sorbitanmonolaurate | none | 130-140 | 70 |
| | | | 5 | | | |
| B | 5 | 85 | Sorbitanmonolaurate | none | 140 | 150 |
| | | | 10 | | | |
| C | 5 | 90 | Sorbitanmonolaurate | none | 140 | 150 |
| | | | 5 | | | |
| D | 5 | 92 | Sorbitanmonolaurate | none | 140-145 | 150 |
| | | | 3 | | | |
| E | 5 | 90 | Tween 80 | none | 140 | 150 |
| | | | 5 | | | |
| F | 5 | 90 | TPGS* | none | 140 | 150 |
| | | | 5 | | | |
| G | 5 | 90 | Propylenglycol monolaurate** | none | 140 | 150 |
| | | | 5 | | | |
| H | 5 | 85 | Propylenglycol monolaurate** | TPGS* | 140 | 150 |
| | | | 6.2 | 3.8 | | |
| I | 5 | 90 | Propylenglycol monolaurate** | TPGS* | 140 | 150 |
| | | | 3.1 | 1.9 | | |
| K | 5 | 89 | Propylenglycol monolaurate** | TPGS* | 140 | 150 |
| | | | 4 | 2 | | |

*tocopheryl polyethylene glycol 1000 succinate
**Type 1

### Example 2

### Bioavailability Evaluation

Protocol for the oral bioavailability studies For bioavailability evaluation, extrudates as obtained in Example 1 were milled and filled into capsules. Each capsule contained 25 mg ABT 869.

The studies were run with liquid clinical formulation as reference (4.0% by weight ABT 869 in ethanol-surfactant solution) in a two-treatment, two-period crossover study.

Dogs (beagle dogs, mixed sexes, weighing approximately 10 kg) received a balanced diet with 27% fat and were permitted water ad libitum. Each dog received a 100 μg/kg subcutaneous dose of histamine approximately 30 minutes prior to dosing. A single dose corresponding to 25 mg ABT 869 was administered to each dog. The dose was

followed by approximately 10 milliliters of water. Blood samples were obtained from each animal prior to dosing and 0.25, 0.5, 1.0, 1.5, 2, 3, 4, 6, 8, 10, 12 and 24 hours after drug administration. The plasma was separated from the red cells by centrifugation and frozen (−30° C.) until analysis. Concentrations of ABT 869 inhibitors were determined by reverse phase HPLC with low wavelength UV detection following liquid-liquid extraction of the plasma samples. The area under the curve (AUC) was calculated by the trapezoidal method over the time course of the study. Each dosage form was evaluated in a group containing 5-6 dogs; the values reported are averages for each group of dogs.

TABLE 2
Results of dog studies with a crossover study design

| | Cmax | Tmax | Pt. Estimate | Pt. Estimate |
|---|---|---|---|---|
| Example | [ [symbol] g/ml] | [h] | [symbol] * | AUC* |
| N | 0.77 | 1.1 | 0.83 | 0.82 |
| G | 0.51 | 1.0 | 1.04 | 1.11 |
| I | 0.46 | 1.4 | 0.93 | 1.07 |
| K | 0.56 | 1.9 | 0.68 | 0.8 |
| R | 0.84 | 1.1 | 1.04 | 1.04 |

*The values are reported as relative bioavailability compared to the bioavailability of the liquid clinical formulation as reference. [symbol]
indicates data missing or illegible when filed

### Example 3

### Manufacture of Tablets

Following the procedure of example 1, an extrudate was obtained from the solid dispersion product ingredients listed in table 3 below. The extrudate was allowed to cool.

The solidified extrudate was milled and the powder was blended with the tabletting excipients listed in table 3. A tablet press was used to prepare tablets containing 2.5 mg or 10 mg, respectively, of ABT-869.

TABLE 3
Tablet composition

| Ingredient | % (w/w) |
|---|---|
| Solid dispersion product | |
| ABT-869 ethanolate | 2.50 |
| Kollidon VA64 | 39.75 |
| Propylene glycol monolaurate (Type I) | 5.00 |
| Vitamin E-TPGS | 2.50 |
| Colloidal silicon dioxide, Type Aerosil 200 | 0.25 |
| Tabletting excipients | |
| Mannitol | 48.50 |
| Colloidal silicon dioxide, Type Aerosil 200 | 1.00 |
| Sodium stearyl fumarate | 0.50 |

### Example 4

### Estimating Pharmacokinetics in Humans

Tablets containing a 10 mg dose of ABT-869 ethanolate, as prepared above, were administered to 11 patients in the morning with 240 mL of water under fasting conditions (defined as no food or drink consumption except water and concomitant medications for 2 hours prior to dosing). Following dosing, 4-mL blood samples were collected for pharmacokinetic analyses at the following times: 0 (pre-dose), 1, 3, 4, 6, 8, 24 and 48 hours. These samples were analyzed for ABT-869 plasma concentrations using Liquid Chromatography/Tandem Mass Spectrometry (LCMS/MS). The lower limit of quantification (LLOQ) for the assay was 1.1 ng/mL.

Pharmacokinetic parameters including the maximum observed plasma concentration _{(Cmax)}, time to Cmax (Tmax), the area under the plasma concentration-time curve (AUC) from 0 to time of the last measurable concentration _{(AUC0-48)} and AUC from 0 to infinite time (AUCc∞) were determined by non-compartmental methods using WinNonlin Professional version 5.2 software. AUC0-48 per mg of dose was 0.40±0.10 μg·h/mL/mg (mean±SD) while AUCc∞ per mg of dose was 0.55±0.17 μg·h/mL/mg. _{Cmax} per mg of dose was calculated to be 0.023±0.004 μg/mL/mg. The ABT-869 tablet has a _{Tmax} of 2.8±0.6 h. The intersubject variability in the ABT-869 tablet was 17% in _{Cmax} and 25% in AUC0-48.

### Example 5

### Comparison of Solid Tumors and Hematological Malignancies

### Pharmacokinetic Parameters; Additional Safety and Efficacy Data

The objective of this analysis was to compare pharmacokinetic (PK) parameters and to monitor safety and early efficacy of ABT-869 when treating solid tumors versus hematologic malignances, Asian versus Caucasian populations and using solution versus tablet formulations.

### Study Designs and Dosing

Study M04-710 and M05-756 are ongoing, open-label, multiple-dose escalation trials with ABT-869 administered daily for 21 consecutive days, with dose escalation scheduled in cohorts of 3 subjects. A cohort expanded to 6 subjects if a dose limiting toxicity (DLT) was seen in at least 1 subject in the dose cohort. In both studies dosing was initiated at 10 mg QD, with subjects self-administering ABT-869 orally (admixed with approximately 60 mL of Ensure Plus or an approved alternative) at bedtime. Food was not allowed 2 h before or after dosing. As a portion of trial M04-710, the bioavailability of ABT-869 as a single dose in a 10 mg tablet formulation was determined relative to the solution formulation.

### Eligibility Criteria

### ≧18 Years of Age or Older.

In M04-710, subjects had either a non-hematologic malignancy that is refractory to standard therapies or for which a standard effective therapy does not exist. In M05-756, subjects had relapsed/refractory acute myelogenous leukemia for which no standard effective therapy is anticipated to result in a durable partial or complete remission or poor-risk myelodysplasia patients (including refractory anemia with excess blast or excess blasts in transformation) who are either relapsed/refractory or who refuse or are not eligible for frontline therapy. ECOG score of 0-2 in study of non-hematologic malignancies or 0-3 in AML/MDS study.

### Documented LV Ejection Fraction of ≧50%.

Adequate hematological, renal and hepatic function as follows:
Absolute neutrophil count≧1,000/mL; platelets≧100,000/mm³; and hemoglobin≧9.0 gm/dL (non-hematologic malignancy study).
Serum creatinine of ≦1.5×upper normal limit (ULM) of institution's normal range (both studies) Bilirubin, AST and/or ALT≦1.5×ULN (non-hematologic malignancy study) or ≦2.5 ULN (AML/MDS study) of institution's normal range.
PTT<1.5 ULN and INR<1.5 (AML/MDS study)
Women with child bearing potential and men agreed to use adequate contraception prior to study entry, for the duration of study participation and up to two months following completion of therapy (both studies).

### Samples for Pharmacokinetic Analyses

In study M04-710, blood samples for pharmacokinetic analysis were collected on Days 1 and 15 at the following times: 0 (pre-dose), 0.5, 1, 2, 3, 4, 6, 8, and 24 h (Day 1 only).

In study M05-756, blood samples for pharmacokinetic analysis were collected on Days 1 and 8 at the following times: 0 (pre-dose), 0.5, 1, 2, 3, 4, 6, 8, and 24 h (Day 1 only).
Urine was collected for the 24 hour period following dosing on Day 13 through bedtime on Day 14 (non-hematologic malignancy study) and following dosing after Day 6 and through bedtime on Day 7 (AML/MDS study).
Plasma and urine concentrations of ABT-869 were determined under the supervision of the Drug Analysis Department at Abbott using LCMS/MS.
PK parameters were determined by noncompartmental analysis using WinNonlin Professional Version 5.0.1.

### Safety Profile

The following safety evaluations were performed during the studies: adverse event monitoring, vital signs, ECGs, physical examination and laboratory tests assessments.

### Results

### Pharmacokinetic Profile

For Study M04-710, the average age for patients enrolled in the study was 58±13 years (mean±SD), while average weight and height measurements were 56.3±16.4 kg and 160.3±8.8 cm, respectively. For M05-756, average age, weight and height were 47±18 years, 75.4±18.8 kg and 169.0±9.4 cm, respectively

The mean pharmacokinetic parameters for study M04-710 are given in Table 4
In study M04-710 (N=30), PK were dose-proportional and time-invariant between 0.10 and 0.30 mg/kg.
˜4% of ABT-869 dose was recovered in the urine as unchanged ABT-869 and about 2% was recovered as a carboxylate metabolite (N=4).

TABLE 4
ABT-869 PK parameters from study M04-710

| | ABT-869 | | | |
|---|---|---|---|---|
| Pharmacokinetic | | 10 mg | | |
| Parameter (Units) | 0.10 mg/kg | (~0.20 mg/kg) | 0.25 mg/kg | 0.30 mg/kg |
| | Study Day 1 | | | |
| N | 11 | 6 | 12 | 3 |
| Tₘₐₓ (h) | 3.5 ± 1.5 | 3.3 ± 1.5 | 2.7 ± 0.8 | 2.0 ± 0.0 |
| Cₘₐₓ (μg/mL) | 0.12 ± 0.05 | 0.21 ± 0.12 | 0.26 ± 0.09 | 0.34 ± 0.09 |
| Cₘₐₓ/D (μg/mL/mg) | 0.020 ± 0.007 | 0.021 ± 0.012 | 0.019 ± 0.006 | 0.020 ± 0.008 |
| AUC_{∞} (μg · h/mL) | 3.1 ± 1.4 | 4.1 ± 2.2 | 5.8 ± 2.9 | 7.9 ± 2.0 |
| AUC_{∞}/D (μg · h/mL/mg) | 0.51 ± 0.21 | 0.41 ± 0.22 | 0.41 ± 0.19 | 0.47 ± 0.19 |
| t_{1/2} (h) | 19.0 ± 5.6 | 14.4 ± 4.6 | 18.9 ± 6.2 | 22.0 ± 2.4 |
| CL (L/h) | 2.3 ± 0.9 | 3.0 ± 1.4 | 3.0 ± 1.3 | 2.4 ± 0.8 |
| | Study Day 15 | | | |
| N | 11 | 6 | 11 | 3 |
| Tₘₐₓ (h) | 3.7 ± 1.5 | 3.0 ± 0.0 | 3.5 ± 1.0 | 3.3 ± 0.6 |
| Cₘₐₓ (μg/mL) | 0.14 ± 0.05 | 0.22 ± 0.17 | 0.31 ± 0.12 | 0.39 ± 0.17 |
| Cₘₐₓ/D (μg/mL/mg) | 0.024 ± 0.008 | 0.026 ± 0.019 | 0.022 ± 0.006 | 0.022 ± 0.008 |
| AUC₀₋₂₄ (μg · h/mL) | 2.1 ± 0.9 | 3.0 ± 1.5 | 4.3 ± 2.1 | 5.3 ± 1.5 |
| AUC₀₋₂₄/D (μg · h/mL/mg) | 0.35 ± 0.15 | 0.35 ± 0.20 | 0.30 ± 0.08 | 0.30 ± 0.07 |

Mean ± SD

TABLE 5
Adjusted Means and Coefficient of Variation (% CV) for PK parameters:
Comparison between Asian versus Caucasian populations

| | | | Dose Normalized | |
|---|---|---|---|---|
| Population | | Dose Normalized | AUC₂₄ | |
| (Race) | N | Cₘₐₓ (μg/mL/mg) | (μg · hr/mL/mg) | T_{1/2} (h) |
| Asian | 37 | 0.018 (37%) | 0.264 (41%) | 18.4 (32%) |
| Caucasian | 12 | 0.016 (27%) | 0.229 (34%) | 14.5 (43%) |

The dose-normalized pharmacokinetic exposures at the steady state were similar between Asian and Caucasian populations for Cₘₐₓ and AUC (p>0.57) after accounting for the effect of the dosing regimens. With the exception of the 10 mg QD dose, Asian patients received dose based on body weight whereas Caucasian patients received a fixed dose. The weight did not have a statistically significant effect for the dose-normalized Cₘₐₓ and AUC values (p>0.20). Since the two populations received various dosing regimens, an analysis was performed for the 10 mg dosing regimen, which was the shared treatment of the two populations. There were no significant race or weight effects (p>0.41) for the dose-normalized Cₘₐₓ and AUC values within the 10 mg dosing regimen.

TABLE 6
Adjusted Means and Coefficient of Variation (%
CV) for PK parameters: Comparison between solid
tumors versus hematological malignancies

| | | | Dose Normalized | |
|---|---|---|---|---|
| | | Dose Normalized | AUC₂₄ | |
| Cancer Type | N | Cₘₐₓ (μg/mL/mg) | (μg · hr/mL/mg) | T_{1/2} (h) |
| Solid | 32 | 0.017 (39%) | 0.273 (40%) | 18.4 (31%) |
| Hematologic | 17 | 0.018 (33%) | 0.230 (39%) | 15.7 (42%) |

The dose-normalized pharmacokinetic exposures at the steady state were similar between the two cancer types for Cₘₐₓ and AUC (p>0.50) after accounting for the effect of the dosing regimens. The weight did not have a statistically significant effect for the dose-normalized Cₘₐₓ and AUC values (p>0.13). Since the subjects in the two cancer types received various dosing regimens, an analysis was performed for 10 mg dosing regimen, which was the common treatment of the two cancer types. Within the 10 mg dosing regimen, there were no significant trend from the cancer types or the effect of weight (p>0.39) for the dose-normalized Cₘₐₓ and AUC values. The comparison between solid tumors and hematologic malignancies was confounded by race because all the data for solid tumors is from the Asian population while most of the data for hematologic malignancies is from the Caucasian population.

TABLE 7
Relative bioavailability: Comparison of
oral solution with solid formulation
PK parameters for solution (0.1 mg/kg) versus
solid formulation (10 mg)

| | | ^{a}DNCₘₐₓ | | DNAUC₀₋₄₈ |
|---|---|---|---|---|
| Formulation | N | (μg/ml/mg) | ^{b}Tₘₐₓ (h) | (μg*hr/mL/mg) |
| Oral Solution | 11 | 0.020 ± 0.007 | 3.0 (3.0-8.0) | 0.40 ± 0.15 |
| Solid form | 11 | 0.023 ± 0.004 | 3.0 (1.0-3.0) | 0.40 ± 0.10 |

Results from study M04-710
^{a}DN = Dose-normalized,
^{b}= median (range),
Mean ± SD

The mean DNAUC₄₈ and DNCₘₐₓ for the oral solution and solid formulation were similar suggesting that the bioavailability of two formulations is similar

### Efficacy in Study M04-710

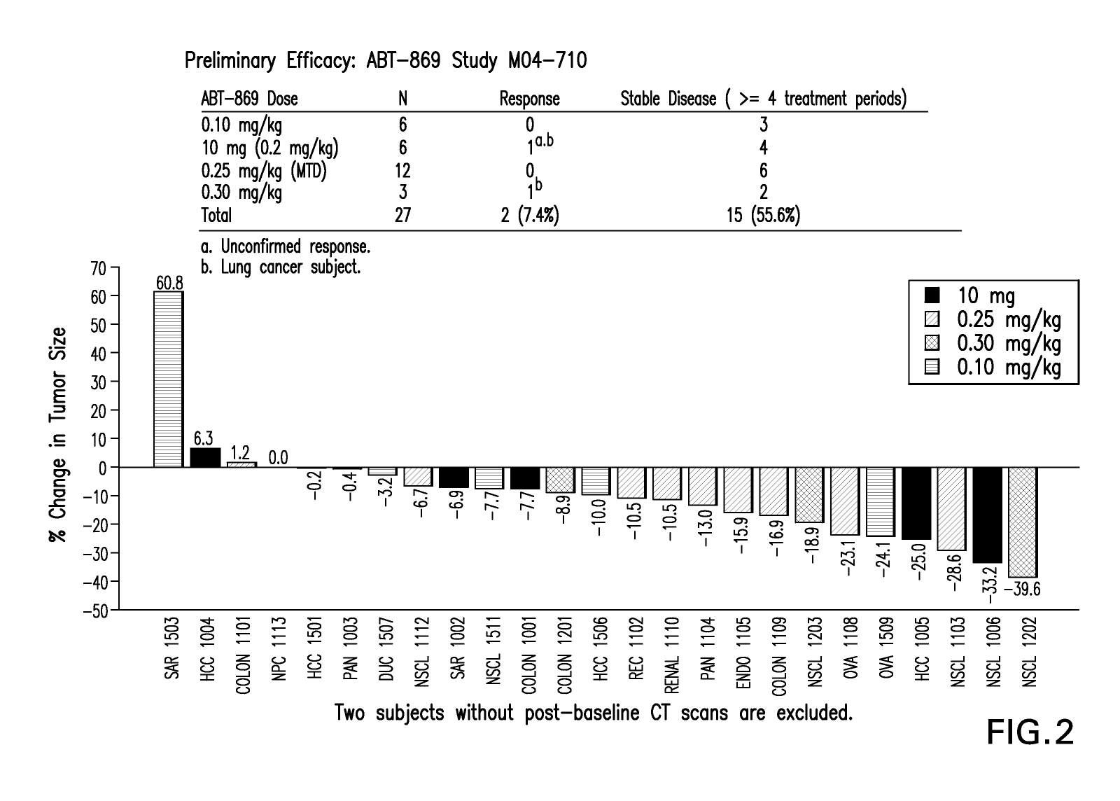

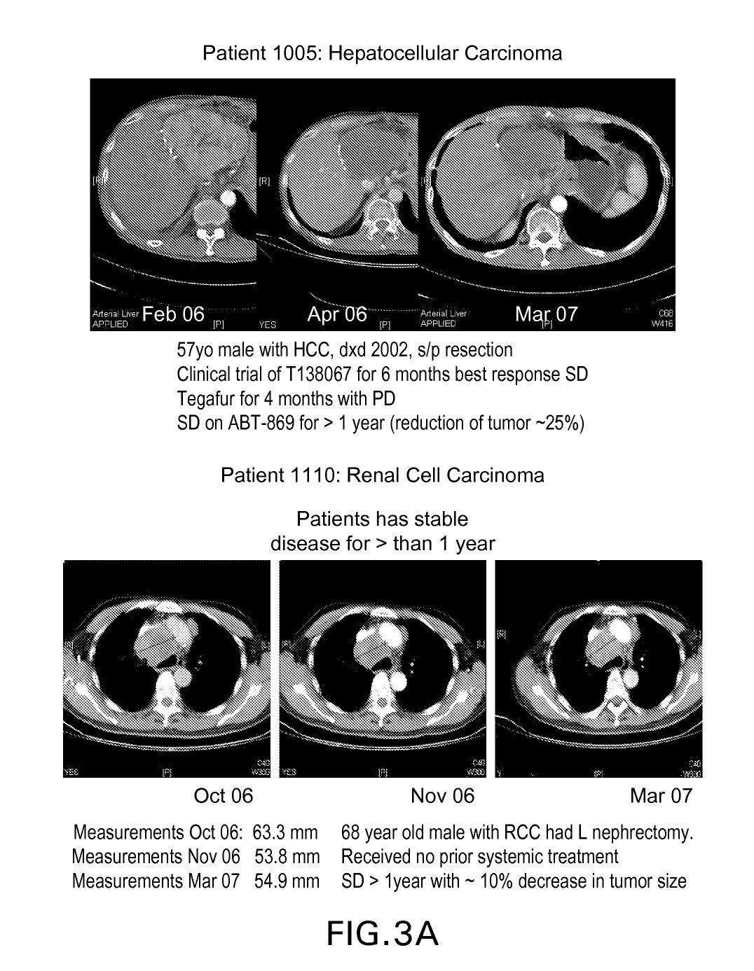

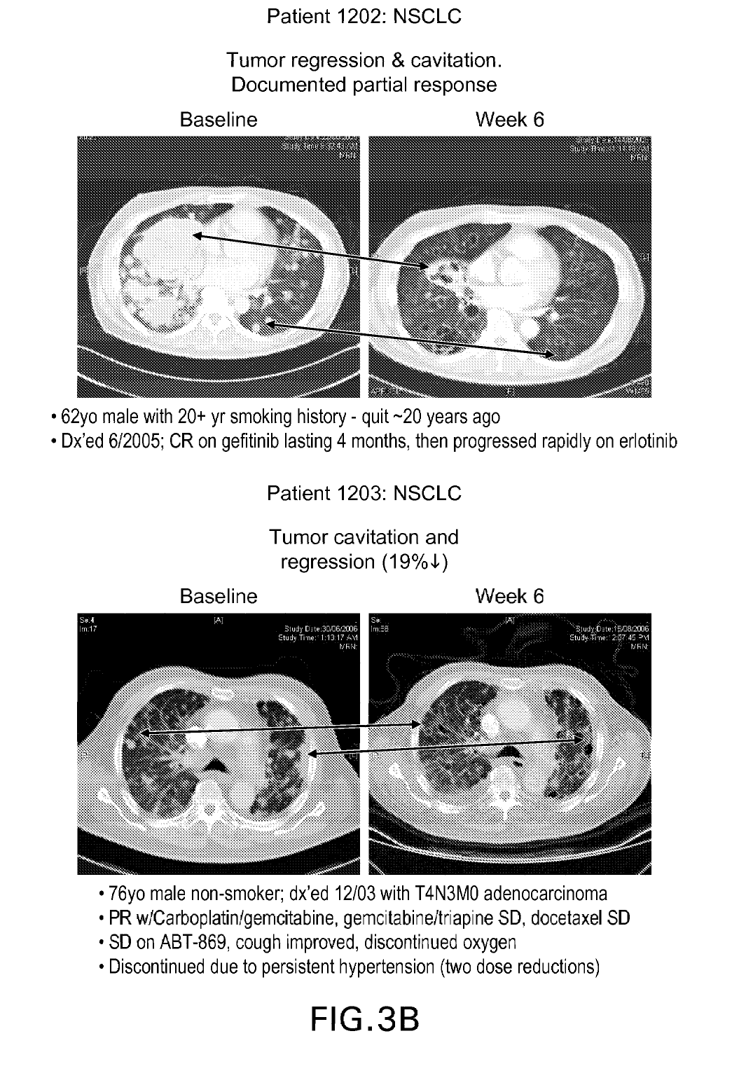

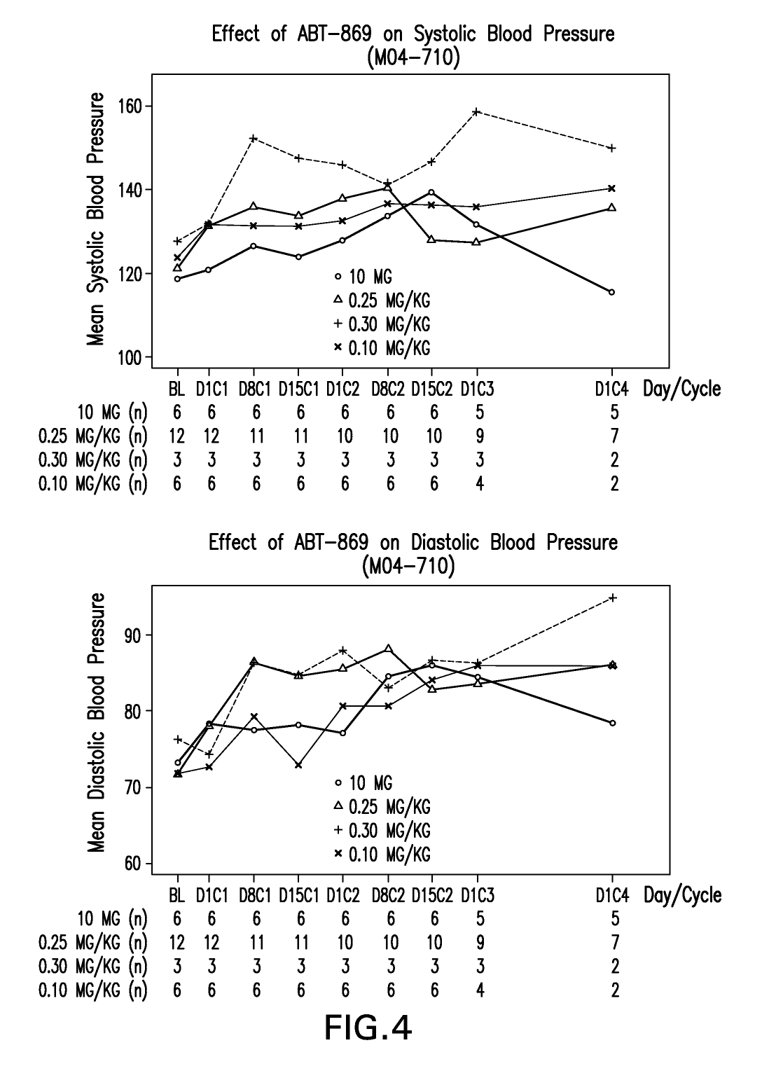

Radiographic changes were observed (FIG. 2, FIG. 3) in an unexpectedly high percentage of patients
Signals of efficacy observed across a broad range of tumors
2 (9%) NSCLC patients experienced partial remission
15 (56%) patients had stable disease (SD) for ≧3 months (4 treatment periods)
6 (22%) patients had SD for ≧6 months, of these patients 4 had SD for >1 year with no accumulation of toxicity
Reductions in tumor size observed in NSCLC, CRC, neuroendocrine tumor, HCC, RCC soft tissue sarcoma and ovarian carcinoma

### Safety

### DLTs Observed:

1 patient treated at 0.10 mg/kg with Grade 3 hypertension
1 patient treated at 10 mg/body with Grade 3 fatigue
1 patient treated at 0.25 mg/kg with Grade 3 proteinuria and 1 patient with Grade 3 hypertension
2 patients treated at 0.30 mg/kg with Grade 3 proteinuria and Grade 3 hypertension.
The most common adverse events (AEs) in study M04-710 (N=27, Table 8) were fatigue, proteinuria, skin related events and hypertension.
The most common AEs in study M05-756 (N=10, Table 9) were diarrhea, febrile neutropenia and fatigue.
Dose limiting fatigue occurred in 3 patients in the 20 mg/body cohort.
In both studies. toxicities were treatable, or reversible with ABT-869 interruption.

TABLE 8
Most Freouently Observed Adverse Events in
all Cycles (Overall, N = 27) (M04-710)

| MedDRA 10.1 Preferred | Grade 1 | Grade 2 | Grade 3/4 | Overall |
|---|---|---|---|---|
| Term | n | n | n | n (%) |
| Fatigue | 14 | 4 | 4** | 22 (81.5) |
| Myalgia | 9 | 6 | 0 | 15 (55.6) |
| Skin Related Event* | 5 | 9 | 1 | 15 (55.6) |
| Hypertension | 0 | 8 | 4 | 12 (44.4) |
| Oral Pain | 9 | 2 | 0 | 11 (40.7) |
| Diarrhoea | 6 | 1 | 0 | 7 (25.9) |
| Anorexia | 6 | 1 | 0 | 7 (25.9) |
| Constipation | 0 | 6 | 0 | 6 (22.2) |
| Arthralgia | 5 | 1 | 0 | 6 (22.2) |
| Proteinuria | 0 | 2 | 4 | 6 (22.2) |
| Nausea | 3 | 2 | 0 | 5 (18.5) |
| Vomiting | 4 | 1 | 0 | 5 (18.5) |

*Skin related event includes Skin Reaction, Skin Lesion, and Rash.
**Grade 3/4 events of fatigue occurred in cycle 3 or later.
Table depicts the highest grade for the same events reported by each subject.

TABLE 9
Most Frequently Observed Adverse Events (>=20%)
in all Cycles (Overall, N = 10) (M05-756)

| MedDRA 10.1 Preferred | Grade 1 | Grade 2 | Grade 3/4 | Overall |
|---|---|---|---|---|
| Term | n | n | n | n (%) |
| Diarrhoea | 2 | 1 | 1 | 4 (40.0) |
| Febrile Neutropenia | 0 | 0 | 3 | 3 (30.0) |
| Asthenia | 1 | 0 | 2 | 3 (30.0) |
| Pyrexia | 1 | 1 | 1 | 3 (30.0) |
| Cough | 3 | 0 | 0 | 3 (30.0) |
| Constipation | 2 | 0 | 0 | 2 (20.0) |
| Nausea | 2 | 0 | 0 | 2 (20.0) |
| Fatigue | 1 | 1 | 0 | 2 (20.0) |
| Klebsiella Bacteraemia | 0 | 0 | 2 | 2 (20.0) |
| Anorexia | 2 | 0 | 0 | 2 (20.0) |
| Hypocalcaemia | 2 | 0 | 0 | 2 (20.0) |
| Hypokalaemia | 2 | 0 | 0 | 2 (20.0) |
| Myalgia | 1 | 0 | 1 | 2 (20.0) |
| Haematuria | 1 | 1 | 0 | 2 (20.0) |
| Alopecia | 0 | 2 | 0 | 2 (20.0) |
| Palmar-plantar | 2 | 0 | 0 | 2 (20.0) |
| erythrodysaesthesia | | | | |
| syndrome | | | | |

Table depicts the highest grade for the same events reported by each subject.

### Conclusions

The time to maximum plasma concentration was approximately 3 h and the mean terminal elimination half-life ranged from 13.9 to 23.1 h in 42 patients across studies.

Dose-normalized PK parameters at the steady state (DNCₘₐₓ, DNAUC₂₄) are similar between Asian versus Caucasian populations and patients with solid tumors versus those with hematological malignancies.

Bioavailability of tablet formulation is similar to the oral solution

Based on the currently available PK data from study M04-710, pharmacokinetics of ABT-869 appears to be approximately dose-proportional (0.10-0.30 mg/kg QD, oral) and time independent with minimal accumulation at day 15

In study M04-710, efficacy is promising with radiographic changes in an unexpectedly high percentage of patients and signals of efficacy observed across a broad range of tumors (NSCLC, CRC, neuroendocrine tumor, HCC, RCC soft tissue sarcoma, ovarian carcinoma).
15 (56%) of patients experienced SD for ≧3 months (4 treatment periods)
6 (22%) of patients experienced SD for ≧6 months, of these patients 4 had SD for >1 year with no accumulation of toxicity
2 patients (9%) with NSCLC experienced a partial response.

Toxicity profile appears to be acceptable
In study M04-710, hypertension, proteinuria and fatigue are dose-limiting
In study M05-756, fatigue is dose-limiting
   All observed toxicities are readily reversible.

We claim:

1. A pharmaceutical dosage form which comprises a solid dispersion product of at least one tyrosine kinase inhibitor, at least one pharmaceutically acceptable polymer, and at least one pharmaceutically acceptable solubilizer.

2. The dosage form of claim 1, which upon contact with an aqueous liquid releases particles having an average particle size of less than about 1000 nm, the particles containing solubilised tyrosine kinase inhibitor.

3. The dosage form of claim 1, wherein the pharmaceutically acceptable solubilizer is selected from the group consisting of polyol fatty acid esters, polyalkoxylated polyol fatty acid esters, polyalkoxylated fatty alcohol ethers, tocopheryl compounds or mixtures of two or more thereof.

4. The dosage form of claim 1, wherein the pharmaceutically acceptable solubilizer has an HLB value in the range of from 3.5 to 13.

5. The dosage form of claim 1, comprising a combination of two or more pharmaceutically acceptable solubilizers.

6. The dosage form of claim 5, wherein the combination of pharmaceutically acceptable solubilizers has an averaged HLB value in the range of from 4.5 to 12.

7. The dosage form of claim 5, wherein the combination of pharmaceutically acceptable solubilizers comprises (i) at least one solubilizer having an HLB value in the range of from 8 to 15 and (ii) at least one solubilizer having an HLB value in the range of from 3 to 6.

8. The dosage form of claim 7, wherein the combination of pharmaceutically acceptable solubilizers comprises (i) at least one tocopheryl compound having a polyalkylene glycol moiety and (ii) at least one alkylene glycol fatty acid monoester or mixture of alkylene glycol fatty acid mono- and diester.

9. The dosage form of claim 8, wherein the tocopheryl compound is alpha tocopheryl polyethylene glycol succinate.

10. The dosage form of claim 8, wherein the alkylene glycol fatty acid monoester is propylene glycol monolaurate.

11. The dosage form of claim 8, wherein the weight ratio of tocopheryl compound and alkylene glycol fatty acid ester is in the range of from 9:1 to 1:9.

12. The solid dosage form of claim 1, wherein said pharmaceutically acceptable polymer is a homopolymer or copolymer of N-vinyl pyrrolidone.

13. The solid dosage form of claim 1, wherein said pharmaceutically acceptable polymer is a copolymer of N-vinyl pyrrolidone and vinyl acetate.

14. The dosage form of claim 1, wherein said tyrosine kinase inhibitor is selected from the group consisting of sorafenib, dasatinib, lapatinib, imatinib, motesanib, vandetanib, MP-412, lestaurtinib, XL647, XL999, tandutinib, PKC412, nilotinib, AEE788, OSI-930, OSI-817, sunitinib maleate, axitinib, N-[4-(3-amino-1H-indazol-4-yl)phenyl]-N′-(2-fluoro-5-methylphenyl)urea (ABT869); N-(4-(4-aminothieno[2,3-d]pyrimidin-5-yl)phenyl)-N′-(2-fluoro-5-(trifluoromethyl)-phenyl)urea; or salts or hydrates or solvates thereof, or combinations thereof.

15. The dosage form of claim 1, wherein said tyrosine kinase inhibitor is poorly water-soluble.

16. The dosage form of claim 1, wherein said tyrosine kinase inhibitor comprises at least one urea moiety in its molecular structure.

17. The solid dosage form of claim 1, containing at least one additive selected from flow regulators, disintegrants, bulking agents and lubricants.

18. The dosage form of claim 1, wherein the solid dispersion product comprises from about 0.5 to 40% by weight of said at least one tyrosine kinase inhibitor, 40 to 97.5% by weight of said at least one pharmaceutically acceptable polymer, 2 to 20% by weight of said at least one solubilizer, and 0 to 15% by weight of additives.

19. The dosage form of claim 1 wherein the solid dispersion product is a melt-processed, solidified mixture.

20. The dosage form of claim 1 wherein the solid dispersion product is obtained by dissolving the at least one tyrosine kinase inhibitor, the at least one pharmaceutically acceptable polymer and the at least one pharmaceutically acceptable solubilizer in a common solvent or combination of solvents and evaporating the solution obtained.

21. The dosage form of claim 1 wherein said tyrosine kinase inhibitor is N-[4-(3-amino-1H-indazol-4-yl)phenyl]-N′-(2-fluoro-5-methylphenyl)urea (ABT 869), the dosage form, when administered to a human patient, producing a plasma profile characterized by a Cₘₐₓ for ABT 869 from about 0.015 μg/mL/mg to about 0.027 μg/mL/mg after a single dose.

22. The dosage form of claim 1 wherein said tyrosine kinase inhibitor is N-[4-(3-amino-1H-indazol-4-yl)phenyl]-N′-(2-fluoro-5-methylphenyl)urea (ABT 869), the dosage form, when administered to a human patient, producing a plasma profile characterized by a Tₘₐₓ for ABT 869 from 1 to about 3 hours after a single dose.

23. The dosage form of claim 1 wherein said tyrosine kinase inhibitor is N-[4-(3-amino-1H-indazol-4-yl)phenyl]-N′-(2-fluoro-5-methylphenyl)urea (ABT 869), the dosage form, when administered to a human patient, producing a plasma profile characterized by a AUC₀₋₄₈ per mg of ABT 869 from about 0.23 μg*hr/mL/mg to about 0.56 μg*hr/mL/mg per mg of dose after a single dose.

24. The dosage form of claim 1 wherein said tyrosine kinase inhibitor is N-[4-(3-amino-1H-indazol-4-yl)phenyl]-N′-(2-fluoro-5-methylphenyl)urea (ABT 869), the dosage form, when administered to a human patient, producing a plasma profile characterized by a AUC0_{-∞} per mg of ABT 869 from about 0.27 μg*hr/mL/mg to about 0.81 μg*hr/mL/mg per mg of dose after a single dose.

25. A method of treating proliferative disorders, comprising administering the dosage form of claim 1 to a subject in need thereof.

26. The method of claim 25, wherein the proliferative disorder is selected from tumors or cancers.

27. The method of claim 25, wherein the proliferative disorder is selected from the
group consisting of neurofibromatosis, tuberous sclerosis, hemangiomas and lymphangiogenesis, cervical, anal and oral cancers, eye or ocular cancer, stomach cancer, colon cancer, bladder cancer, rectal cancer, liver cancer, pancreas cancer, lung cancer, breast cancer, cervix uteri cancer, corpus uteri cancer, ovary cancer, prostate cancer, testis cancer, renal cancer, brain cancer, cancer of the central nervous system, head and neck cancer, throat cancer, skin melanoma, acute lymphocytic leukemia, acute myelogenous leukemia, Ewing's Sarcoma, Kaposi's Sarcoma, basal cell carcinoma and squamous cell carcinoma, small cell lung cancer, choriocarcinoma, rhabdomyosarcoma, angiosarcoma, hemangioendothelioma, Wilms Tumor, neuroblastoma, mouth/pharynx cancer, esophageal cancer, larynx cancer, lymphoma, multiple myeloma; cardiac hypertrophy, age-related macular degeneration and diabetic retinopathy.

28. A method of preparing a solid dosage form of claim 1 which comprises:
a) preparing a homogeneous melt of said at least one tyrosine kinase inhibitor, said at least one pharmaceutically acceptable polymer and said at least one solubilizer, and
b) allowing the melt to solidify to obtain a solid dispersion product.

29. The method of claim 28, additionally comprising grinding said solid dispersion product and compressing said solid dispersion product into a tablet.

30. The method of claim 28, additionally comprising grinding said solid dispersion product and filling said solid dispersion product into a capsule shell.

31. The method of claim 28, wherein the melt is shaped into a film or a foam before being allowed to solidify.
